(19) United States
(12) Patent Application Publication
(10) Pub. No.: US 2016/0113666 A1
(43) Pub. Date: Apr. 28, 2016

(54) METHODS AND APPARATUS FOR TREATING PULMONARY EMBOLISM

(71) Applicant: Inceptus Medical, LLC, Aliso Viejo, CA (US)

(72) Inventors: Richard Quick, Mission Viejo, CA (US); Brian J. Cox, Laguna Niguel, CA (US); Paul Lubock, Monarch Beach, CA (US); Robert F. Rosenbluth, Laguna Niguel, CA (US)

(21) Appl. No.: 14/989,697

(22) Filed: Jan. 6, 2016

Related U.S. Application Data
(63) Continuation of application No. 14/299,933, filed on Jun. 9, 2014, now Pat. No. 9,259,237.
(60) Provisional application No. 61/949,953, filed on Mar. 7, 2014.

(51) Int. Cl. A61B 17/221; A61B 17/3207

(52) U.S. Cl. CPC A61B 17/221; A61B 17/320725; A61B 2017/2215; A61B 2017/00292

(57) ABSTRACT

A device and method for intravascular treatment of an embolism, and particularly a pulmonary embolism, is disclosed herein. One aspect of the present technology, for example, is directed toward a clot treatment device that includes a support member configured to extend through a delivery catheter and a plurality of clot engagement members positioned about the circumference of a distal portion of the support member. The individual clot engagement members can have a first portion and a second portion extending from the first portion, and the first portions can have a proximal region attached to the support member. In the deployed state, the individual second portions can extend from the distal region of one of the first portions and project radially outwardly relative to the support member in a curve that has a proximally extending section which defines a proximally facing concave portion.

### CROSS-REFERENCE TO RELATED APPLICATIONS

The present application claims the benefit of U.S. Provisional Patent Application No. 61/949,953 filed Mar. 7, 2014, entitled “METHODS AND APPARATUS FOR TREATING EMBOLISM,” which is incorporated herein by reference in its entirety.

### TECHNICAL FIELD

The present technology relates generally to devices and methods for intravascular treatment of emboli within a blood vessel of a human patient. Many embodiments of the technology relate to the intravascular treatment of a pulmonary embolism.

### BACKGROUND

Thromboembolism occurs when a thrombus or blood clot trapped within a blood vessel breaks loose and travels through the blood stream to another location in the circulatory system, resulting in a clot or obstruction at the new location. As shown schematically in FIG. 1, when a clot C forms in the venous circulation V, it often travels to the lungs L via the heart H and lodges within a pulmonary blood vessel PV causing a pulmonary embolism PE. A pulmonary embolism PE can decrease blood flow through the lungs L, which in turn causes decreased oxygenation of the lungs L, heart H and rest of the body. Moreover, pulmonary embolisms can cause the right ventricle RV of the heart H to pump harder to provide sufficient blood to the pulmonary blood vessels PV, which can cause right ventricle RV dysfunction (dilation), and heart failure in more extreme cases.

Conventional approaches to treating thromboembolism and/or pulmonary embolism include clot reduction and/or removal. For example, anticoagulants can be introduced to the affected vessel to prevent additional clots from forming, and thrombolytics can be introduced to the vessel to at least partially disintegrate the clot. However, such agents typically take a prolonged period of time (e.g., hours, days, etc.) before the treatment is effective and in some instances can cause hemorrhaging. Transcatheter clot removal devices also exist, however, such devices are typically highly complex, prone to cause trauma to the vessel, hard to navigate to the pulmonary embolism site, and/or expensive to manufacture. Conventional approaches also include surgical techniques that involve opening the chest cavity and dissecting the pulmonary vessel. Such surgical procedures, however, come with increased cost, procedure time, risk of infection, higher morbidity, higher mortality, and recovery time. Accordingly, there is a need for devices and methods that address one or more of these deficiencies.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a schematic illustration of an embolism traveling through the heart and forming an embolism in a pulmonary vessel.

FIG. 2A is a perspective view of one embodiment of a clot treatment device in a collapsed or delivery state configured in accordance with an embodiment of the present technology.

FIG. 2B is a perspective view of the clot treatment device of FIG. 2A in a deployed state configured in accordance with an embodiment of the present technology.

FIG. 2C is an enlarged view of a portion the clot treatment device shown in FIG. 2A.

FIG. 2D is an axial-perspective view of a portion of the clot treatment device shown in FIG. 2A.

FIGS. 3A-3C are isolated, enlarged side views of clot engagement members in a deployed state configured in accordance with embodiments of the present technology.

FIG. 4A is a perspective view of another embodiment of a clot treatment device in a collapsed or delivery state configured in accordance with an embodiment of the present technology.

FIG. 4B is a perspective view of the clot treatment device of FIG. 4A in a deployed state configured in accordance with an embodiment of the present technology.

FIG. 5 is a perspective view of a clot treatment device configured in accordance with another embodiment of the present technology.

FIG. 6 is a perspective view of a clot treatment device configured in accordance with another embodiment of the present technology.

FIG. 7A is a perspective view of a clot treatment device configured in accordance with another embodiment of the present technology.

FIG. 7B is a cross-sectional end view taken along line 7B-7B in FIG. 7A.

FIG. 8 is a perspective view of a clot treatment device configured in accordance with another embodiment of the present technology.

FIG. 9A is a perspective view of a clot treatment device configured in accordance with another embodiment of the present technology.

FIG. 9B is a cross-sectional end view of a portion of the clot treatment device shown in FIG. 9A.

FIG. 9C is a side view of a binding member configured in accordance with the present technology.

FIG. 10 is a side partial cross-sectional view of a delivery system configured in accordance an embodiment of the present technology.

FIGS. 11A-11K illustrate a method for using a clot treatment device configured in accordance with the present technology to remove clot material from a vessel.

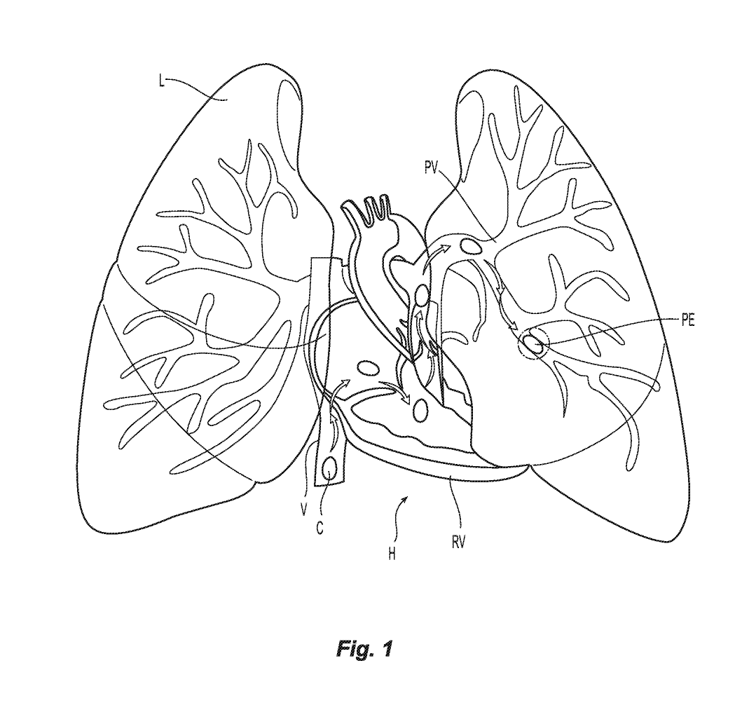

### DETAILED DESCRIPTION

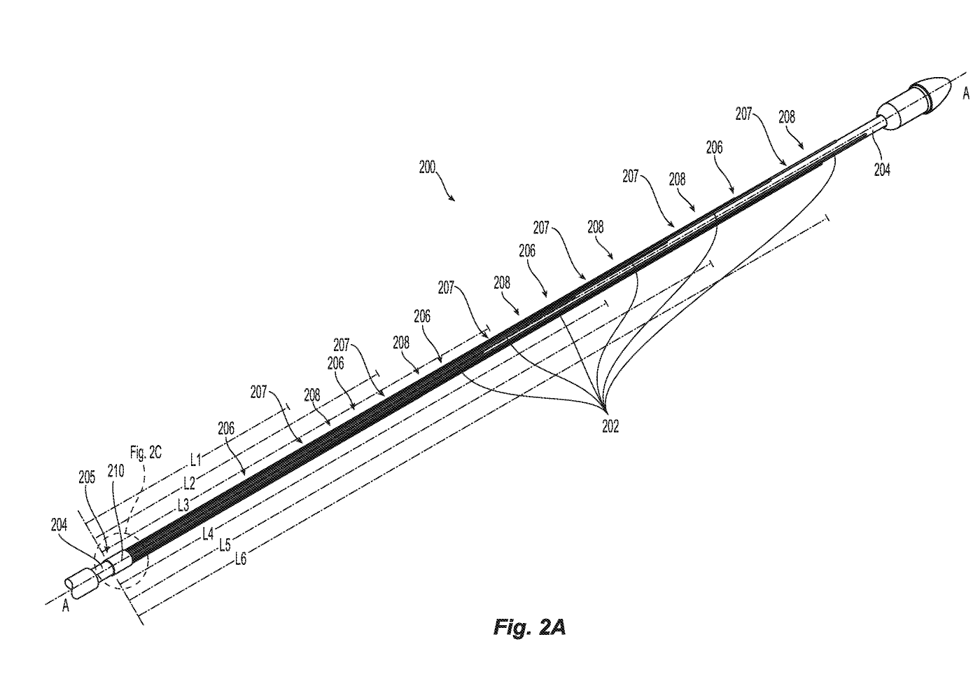

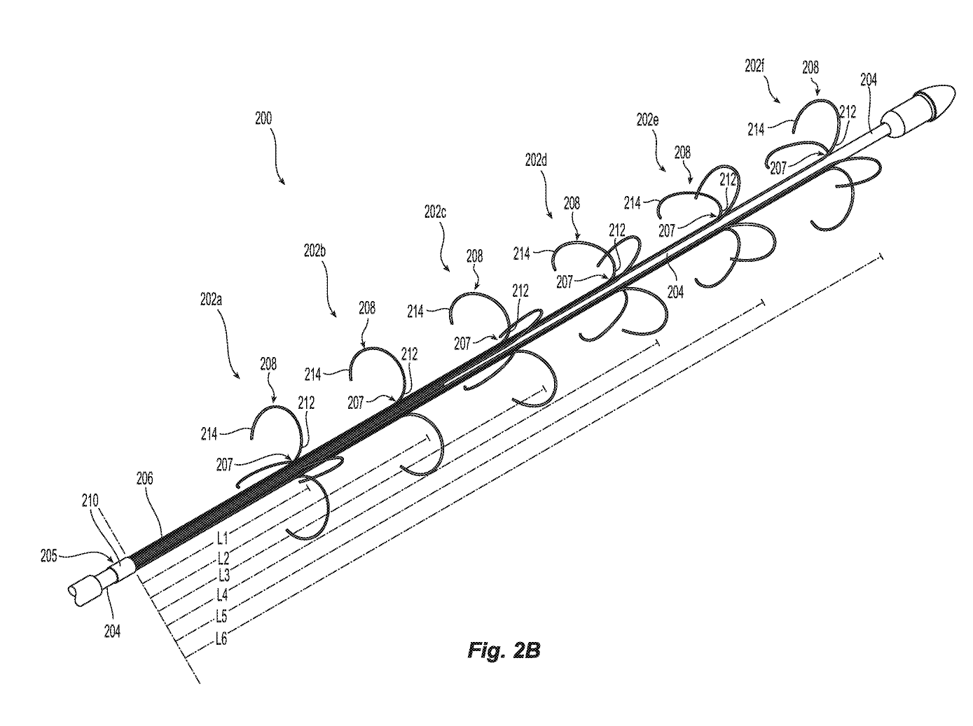

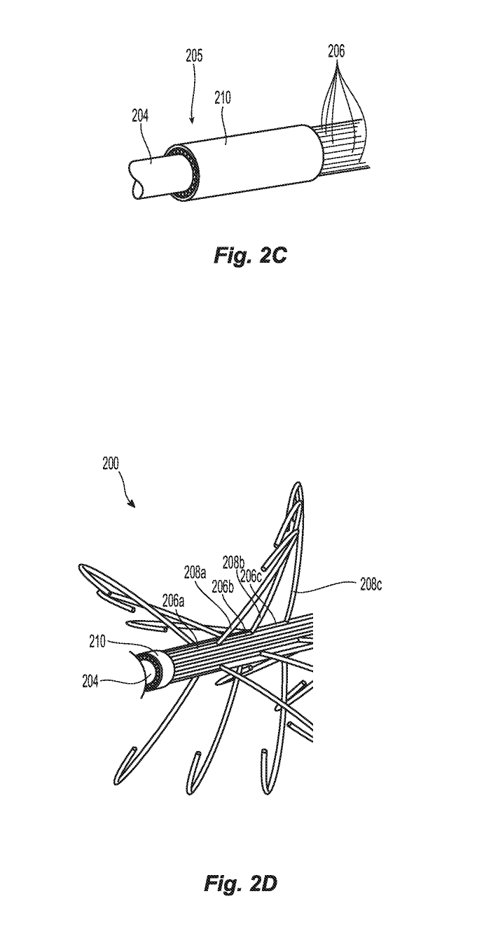

Specific details of several embodiments of clot treatment devices, systems and associated methods in accordance with the present technology are described below with reference to FIGS. 2A-11K. Although many of the embodiments are described below with respect to devices, systems, and methods for treating a pulmonary embolism, other applications and other embodiments in addition to those described herein are within the scope of the technology. Additionally, several other embodiments of the technology can have different states, components, or procedures than those described herein. Moreover, it will be appreciated that specific elements, substructures, advantages, uses, and/or other features of the embodiments described with reference to FIGS. 2A-11K can be suitably interchanged, substituted or otherwise configured with one another in accordance with additional embodiments of the present technology. Furthermore, suitable elements of the embodiments described with reference to FIGS. 2A-11K can be used as standalone and/or self-contained devices. A person of ordinary skill in the art, therefore, will accordingly understand that the technology can have other embodiments with additional elements, or the technology can have other embodiments without several of the features shown and described below with reference to FIGS. 2A-11K.

With regard to the terms “distal” and “proximal” within this description, unless otherwise specified, the terms can reference a relative position of the portions of a clot treatment device and/or an associated delivery device with reference to an operator and/or a location in the vasculature.

### I. Selected Embodiments of Clot Treatment Devices

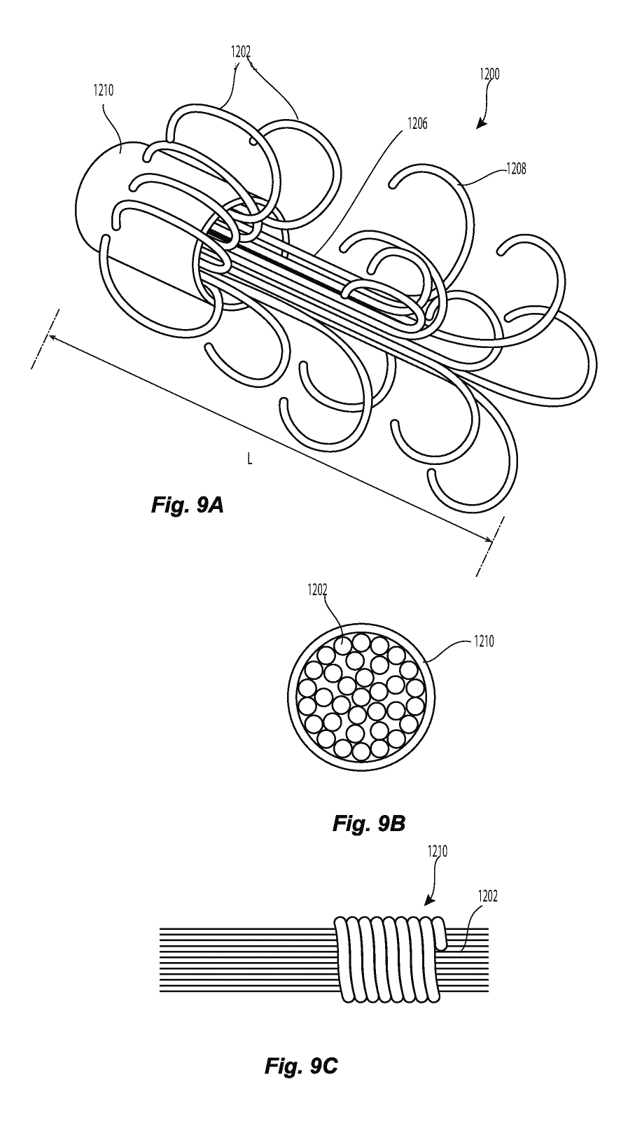

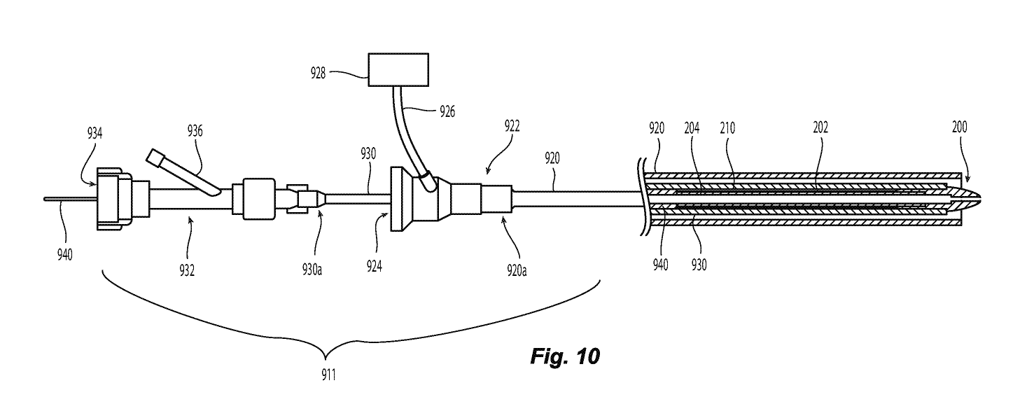

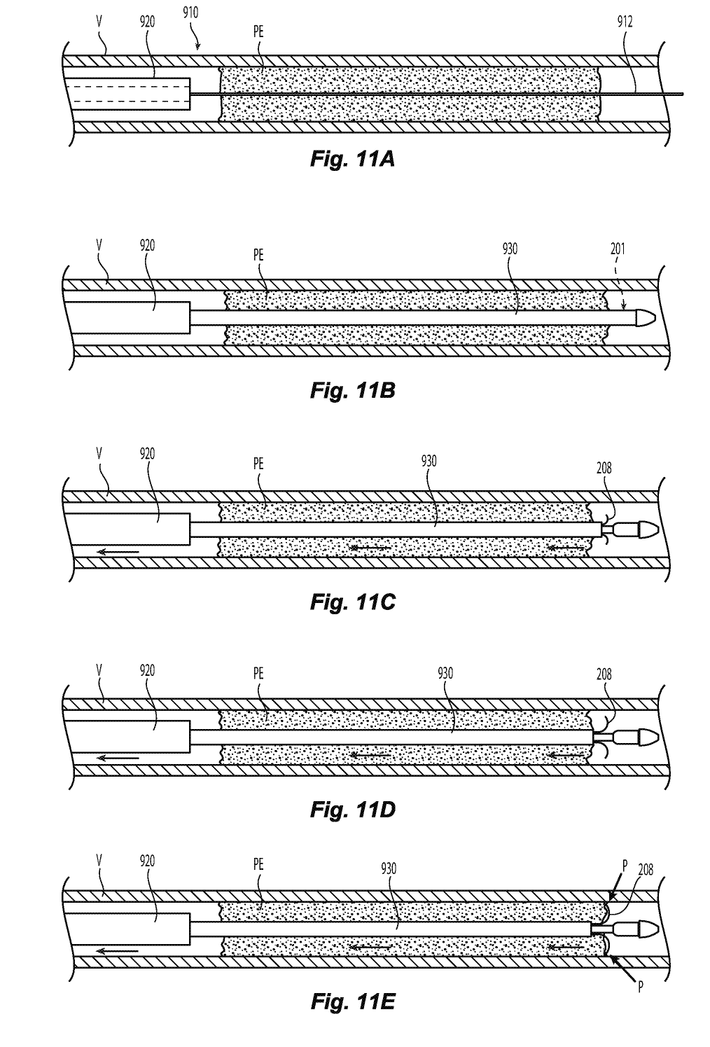

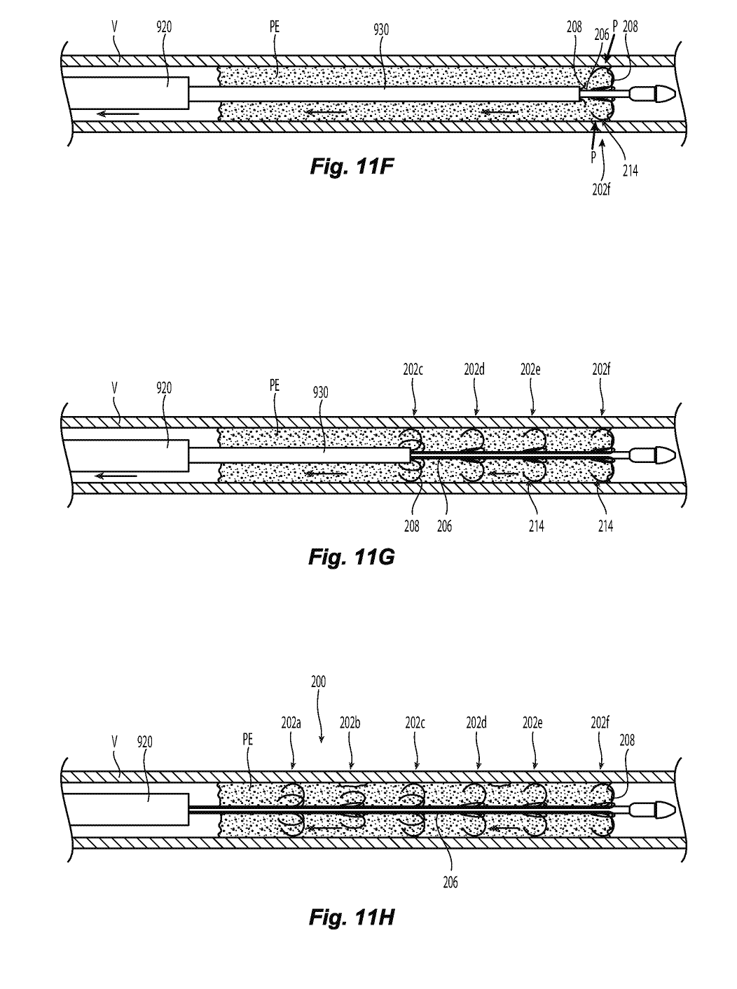

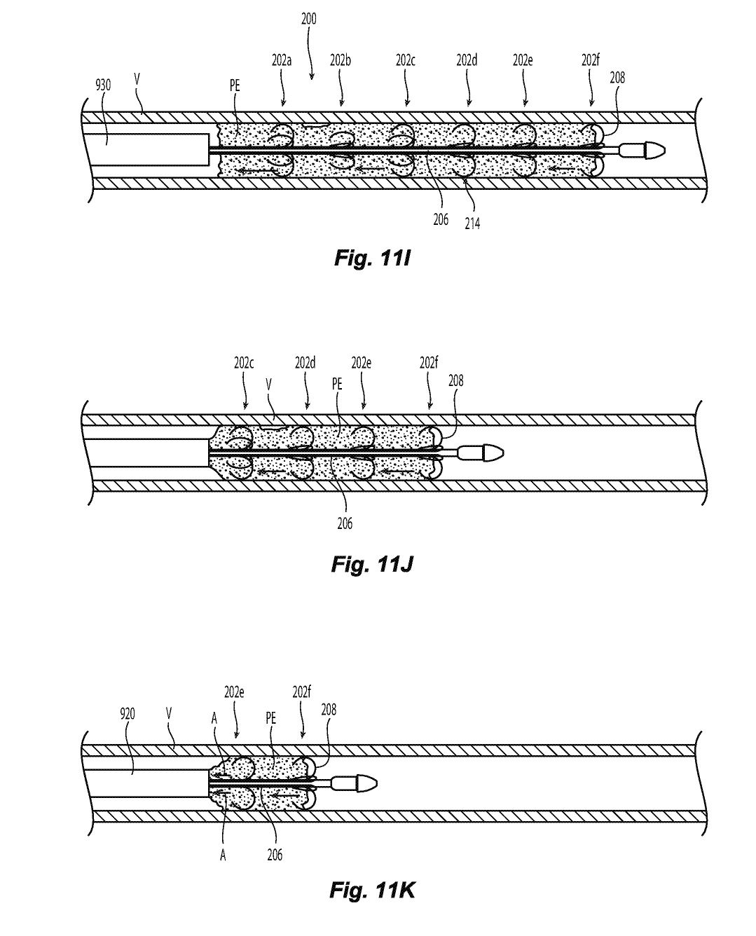

FIG. 2A is a perspective view of one embodiment of a clot treatment device 200 (“the device 200”) in a low-profile or delivery state, and FIG. 2B is a perspective view of the device 200 in an unrestricted expanded or deployed state that is well suited for removing clot material from a blood vessel (e.g., a pulmonary blood vessel). Referring to FIGS. 2A and 2B together, the device 200 can include a support member 204 and a plurality of clot engagement members 202 positioned about the circumference of the support member 204. As best shown in FIG. 2B, the individual clot engagement members 202 can include a first portion 206 having a proximal region 205 and a distal region 207, and a second portion 208 extending from the distal region 207 of the first portion 206. In the delivery state, as shown in FIG. 2A, the clot engagement members 202 can be generally linear and extend generally parallel to the support member 204. In the expanded state, as shown in FIG. 2B, the second portions 208 can project radially outwardly relative to the support member 204 in a curved shape. The second portions 208 can have a proximally facing section 212 which defines a proximally facing concave portion, and, in some embodiments, the second portions 208 can further include an end section 214 that curves radially inwardly from the proximally facing section 212. When deployed within a blood vessel adjacent to clot material, the clot engagement members 202 are configured to penetrate the clot material along an arcuate path and hold clot material to the device 200, as discussed in greater detail below with reference to FIGS. 10-11K.

FIG. 2C is an enlarged view of a portion of the device 200 of FIG. 2A showing that the device 200 can include a hub 210 that couples the proximal regions 205 of the first portions 206 to the support member 204. The first portions 206 can extend distally from their proximal regions 205 in a longitudinal direction along the length of the support member 204 to their distal regions 207, and the distal regions 207 can be free to move relative to the support member 204. As such, the first portions 206 can be cantilevered portions of the clot engagement members 202 that enable the clot engagement members 202 to flex and move independently of the support member 204 in response to forces present within the blood vessel, such as blood flow, gravity, and/or the local anatomy. The first portions 206 can be sufficiently rigid to maintain a generally linear shape along their respective lengths, yet flexible enough to bend and/or flex about the hub 210. For example, in some instances, in response to local forces, one or more of the distal regions 207 of the first portions 206 can be spaced radially apart from the support member 204 such that one or more first portions 206 forms an angle with the support member 204.

Referring back to FIGS. 2A and 2B, the first portions 206 of different clot engagement members 202 can have different lengths such that the second portions 208 of at least two clot engagement members extend radially outwardly at different locations along the length of the support member 204. For example, as best shown in FIG. 2B, the clot treatment device 200 can include a first group 202a of clot engagement members 202 having first portions 206 with a first length L1, a second group 202b of clot engagement members 202 having first portions 206 with a second length L2 greater than the first length L1, a third group of clot engagement members 202c having first portions 206 with a third length L3 greater than the second length L2, a fourth group of clot engagement members 202d having first portions 206 with a fourth length L4 greater than the third length L3, a fifth group of clot engagement members 202e having first portions 206 with a fifth length L5 greater than the fourth length L4, and a sixth group of clot engagement members 202f having first portions 206 with a sixth length L6 greater than the fifth length L5. It will be appreciated that although six groups of clot engagement members are shown in FIGS. 2A and 2B, in other embodiments the clot treatment device can have more or fewer than six groups (e.g., one group, two groups, three groups, seven groups, ten groups, etc.) and/or the lengths of all or some of the first portions 206 can be the same or different.

Moreover, the second portions 208 of the first group 202a of clot engagement members 202 extend radially outward at a first area of the support member 204, the second portions 208 of the second group 202b of the clot engagement members 202 extend radially outward from a second area of the support member 204, the second portions 208 of the third group 202c of clot engagement members 202 extend radially outward from a third area of the support member 204, the second portions 208 of the fourth group 202d of clot engagement members 202 extend radially outward from a fourth area of the support member 204, the second portions 208 of the fifth group 202e of clot engagement members 202 extend radially outward from a fifth area of the support member 204, and the second portions 208 of the sixth group 202f of clot engagement members 202 extend radially outward from a sixth area of the support member 204. It will be appreciated that although six areas of clot engagement members are shown in FIGS. 2A and 2B, in other embodiments the clot treatment device can have more or fewer than six areas (e.g., one area, two areas, three areas, five areas, nine areas, etc.).

FIG. 2D is an enlarged, axial-perspective view of a portion of the device 200 in which the groups of clot engagement members 202a-f (only the first, second and third groups 202a-c shown) are arranged about the circumference of the support member 204 such that the second portions (labeled 208a-c) of adjacent groups 202a-c are circumferentially offset from one another. As such, in the embodiment shown in FIG. 2D, the second portions 208 of adjacent groups of clot engagement members 202a-f are not circumferentially aligned, and thus can engage the clot material at different circumferential positions along the length of the clot material.

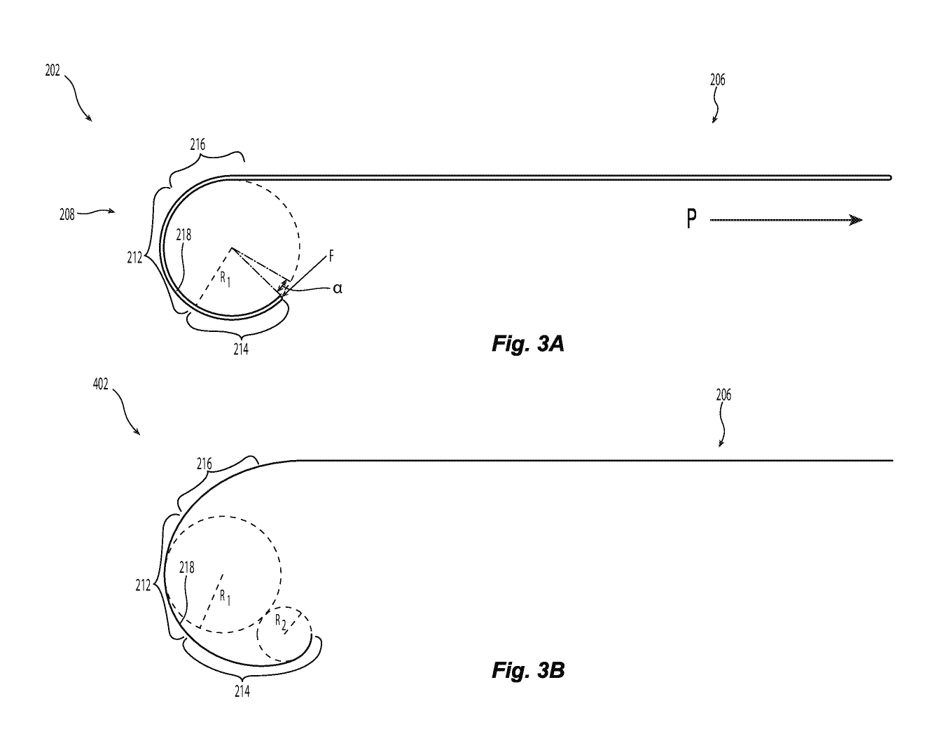

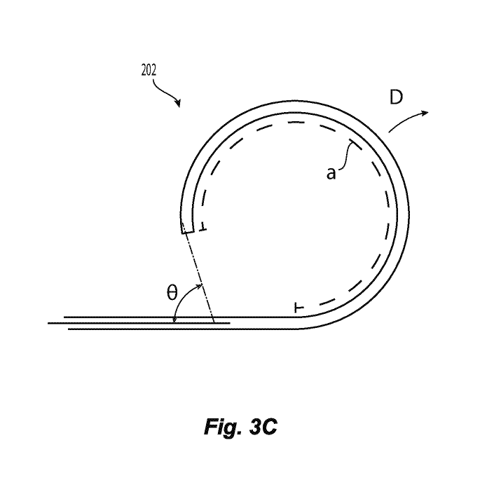

FIG. 3A is a side view of a clot engagement member 202 in the expanded state. Individual clot engagement members can be made from a shape memory material such that, when unconstrained, assume a preformed curved shape. As shown in FIG. 3A, the second portion 208 can have an arcuate shape that includes an outwardly extending section 216, the proximally facing section 212 extending from the outwardly extending section 216, and the end section 214 extending from the proximally facing section 212. In one embodiment, the demarcation between the proximally facing section 212 and the end section 214 occurs at an apex 218 of the second portion 208. The proximally facing section 212 is configured to retain clot material with the clot engagement member 202 as the device 200 is pulled proximally through the vessel (arrow P), and the apex 218 provides a smooth curve that can atraumatically slide along the vessel wall as the device 200 is pulled proximally through the vessel. In the embodiment shown in FIG. 3A, the second portion 208 of the clot treatment device 200 can have a single or constant radius of curvature R₁. In other embodiments, such as the clot engagement member 402 shown in FIG. 3B, the second portions 208 can have a plurality of radii of curvature, such as a first region with a first radius of curvature R₁ and a second region with a second radius of curvature R₂. In the embodiment shown in FIGS. 2A-2D, the second portions 208 of the clot engagement members 202 have a single radius of curvature that is the same for all of the clot engagement members 202. In other embodiments, the device 200 can have a first group of second portions with a constant radius of curvature and a second group of second portions with a plurality of radii of curvature. Moreover, in additional embodiments the device 200 can include a first group of second portions having a first radius of curvature and a second group of second portions having a second radius of curvature different than the first radius of curvature. In some embodiments, the radius R₁ of the clot engagement members 202 can be between about 1.5 mm and about 12 mm, and in some embodiments, between about 2 mm and about 12 mm.

As shown in FIG. 3C, the arc length a of the clot engagement members 202 may be substantially greater than 180 degrees to provide several benefits in performance of clot engagement and retrieval. In particular, a greater arc length a can provide improved clot engagement during retraction when resistance due to clot friction and interference with the vessel wall deflects the clot engagement member 202 distally (arrow D). A greater arc length a may provide more deflection and/or unravelling or straightening of the arcuate shape without loss of engagement with the clot. In some embodiments, the arc length a of the clot engagement members 202 can be greater than about 200 degrees. In some embodiments the arc length a of the clot engagement members 202 may be between about 200 degrees and 340 degrees and between about 240 degrees and 300 degrees in other embodiments. It can be advantageous to keep the arc length a under about 360 degrees so as to avoid overlap of the clot engagement member 202. Greater arc length a can allow for the use of smaller clot engagement member filaments or wires that may be particularly beneficial for minimization of the collapsed profile of the device. Greater arc length a can also allow for a larger total number of clot engagement members 202 that also enhance the ability of the device to remove embolic material from a vessel. Moreover, in some embodiments, the distal end of the clot engagement members 202 may define an angle with respect to the axis of the support member and/or the straight portion of the engagement members (as shown in FIG. 3C). This angle may be between about 30 degrees and about 90 degrees, and in some embodiments between about 40 degrees and about 80 degrees.

The clot engagement members 202 can be made from a variety of materials. In a particular embodiment, the clot engagement members 202 comprise a material with sufficient elasticity to allow for repeated collapse into an appropriately sized catheter and full deployment in a blood vessel. Such suitable metals can include nickel-titanium alloys (e.g., Nitinol), platinum, cobalt-chrome alloys, Elgiloy, stainless steel, tungsten, titanium and/or others. Polymers and metal/polymer composites can also be utilized in the construction of the clot engagement members. Polymer materials can include Dacron, polyester, polyethylene, polypropylene, nylon, Teflon, PTFE, ePTFE, TFE, PET, TPE, PLA silicone, polyurethane, polyethylene, ABS, polycarbonate, styrene, polyimide, PEBAX, Hytrel, polyvinyl chloride, HDPE, LDPE, PEEK, rubber, latex and the like. In some embodiments, the clot engagement members 202 may comprise an environmentally responsive material, also known as a smart material. Smart materials are designed materials that have one or more properties that can be significantly changed in a controlled fashion by external stimuli, such as stress, temperature, moisture, pH, electric or magnetic fields.

In some embodiments, portions of the exterior surfaces of the support member 204 and/or clot engagement members 202 may be textured, or the exterior surfaces can include microfeatures configured to facilitate engagement or adhesion of thrombus material (e.g., ridges, bumps, protrusions, grooves, cut-outs, recesses, serrations, etc.). In some embodiments, the clot engagement members 202 may be coated with one or more materials to promote platelet activation or adhesion of thrombus material. Adhesion of thrombi to clot engagement members 202 may facilitate capture and/or removal.

In some embodiments, the clot treatment device 200 can include between about 8 and about 80 clot engagement members 202, and in some embodiments, between about 12 and about 60 clot engagement members 202. In a particular embodiment, the clot treatment device 200 can include between about 16 and about 40 clot engagement members 202. The clot engagement members 202 can individually have one consistent diameter or have a variety of diameters (among the members 202) along their lengths. In addition, an individual clot engagement member 202 may have a tapered or varying diameter along its length to provide desired mechanical characteristics. The average diameter of the clot engagement members 202 can be between about 0.1 mm to about 0.2 mm in some embodiments and in a particular embodiment, between about 0.12 mm and 0.16 mm.

In any of the embodiments described herein, the clot engagement members 202 can be formed from a filament or wire having a circular cross-section. Additionally, the clot engagement members 202 can be formed from a filament or wire having a non-circular cross-section. For example, filaments or wires having square, rectangular and oval cross-sections may be used. In some embodiments, a rectangular wire (also known as a “flat wire”) may have a height or radial dimension of between about 0.05 mm to about 0.2 mm. In some embodiments, a rectangular wire may have a width or transverse dimension of between about 0.08 mm to about 0.3 mm. In some embodiments, a rectangular wire may have a height to width ratio of between about 0.3 to about 0.9 and between about 1 and about 1.8.

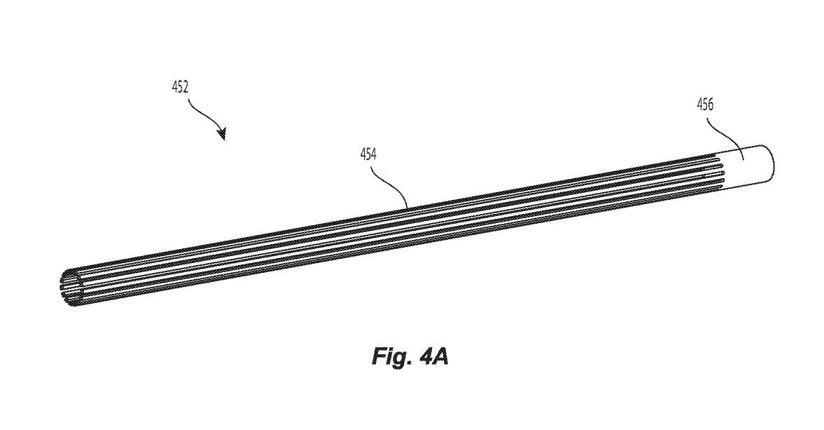

FIGS. 4A and 4B illustrate an embodiment in which clot engagement members having non-circular cross-sections are fabricated from a tube (e.g., a hypotube). The tube may be cut or machined by various means known in the art including conventional machining, laser cutting, electrical discharge machining (EDM) or photochemical machining (PCM). Referring to FIG. 4A, a tube may be cut to form a plurality of clot engagement members 454 that are integral with a hub member 456. The cut tube may then be formed by heat treatment to move from a delivery state shown in FIG. 4A to a deployed state shown in FIG. 4B in which an array of arcuate clot engagement members 454 project radially outward. As is known in the art of heat setting, a fixture or mold may be used to hold the structure in its desired final configuration and subjected to an appropriate heat treatment such that the clot engagement members assume or are otherwise shape-set to the desire arcuate shape. In some embodiments, the device or component may be held by a fixture and heated to about 475-525° C. for about 5-15 minutes to shape-set the structure. In some embodiments, the tubular clot engagement structure may be formed from various metals or alloys such as Nitinol, platinum, cobalt-chrome alloys, 35N LT, Elgiloy, stainless steel, tungsten or titanium.

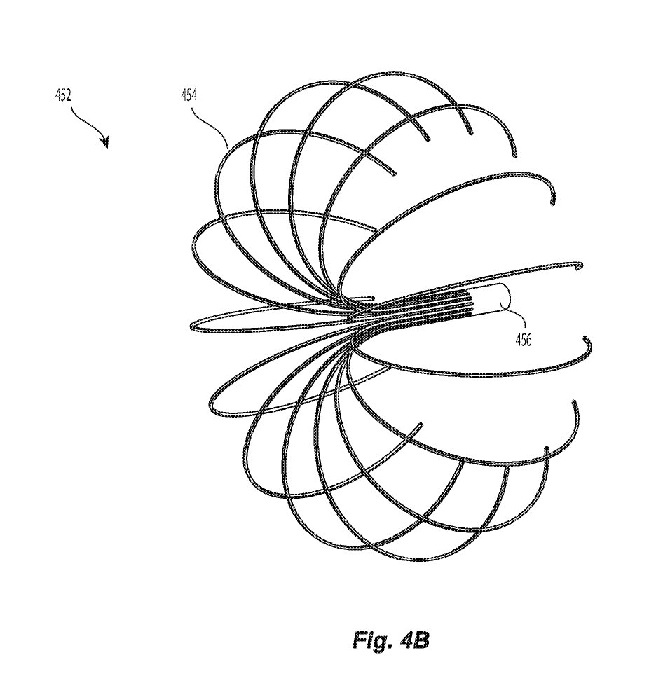

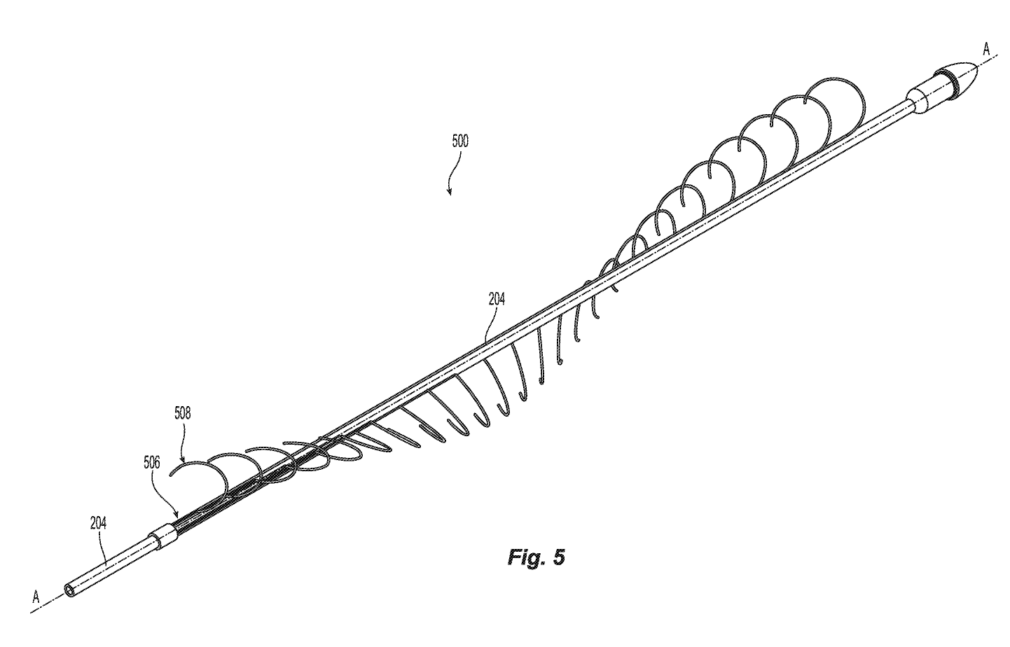

FIG. 5 is a perspective view of another embodiment of a clot treatment device 500 in a deployed state in accordance with the present technology. As shown in FIG. 5, the clot treatment device 500 can include a plurality of clot engagement members 502 generally similar to the clot engagement members 202 and 402 described with reference to FIGS. 2A-4B, except the clot engagement members 502 of FIG. 5 are arranged about the support member 204 such that the length of the first portions 506 increase in a clockwise or counterclockwise direction about 360 degrees of the support member 204. As such, the second portions 508 spiral around the length of the support member 204 and each successive second portion 508 extends from a location along the shaft that is circumferentially offset and distal to the location of the immediately adjacent second portion 508.

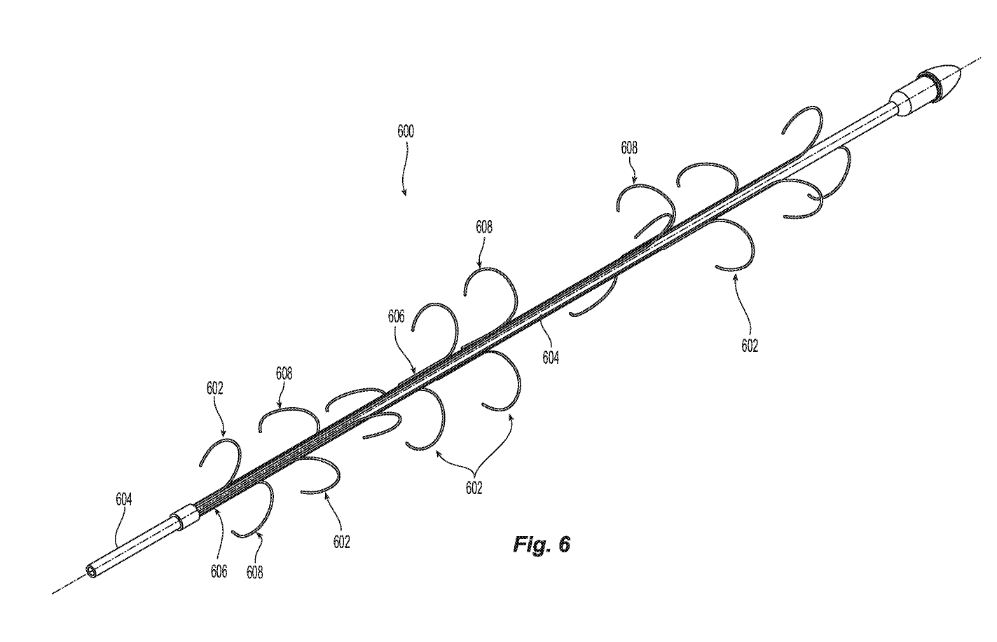

FIG. 6 is a perspective view of another embodiment of a clot treatment device 600 in a deployed state in accordance with the present technology. The clot treatment device 600 can include a plurality of clot engagement members 602 generally similar to the clot engagement members 202 and 402 described with reference to FIGS. 2A-4B, except the second portions 608 of the clot engagement members 602 of FIG. 6 are not arranged in groups, but instead extend at irregular intervals from support member 204.

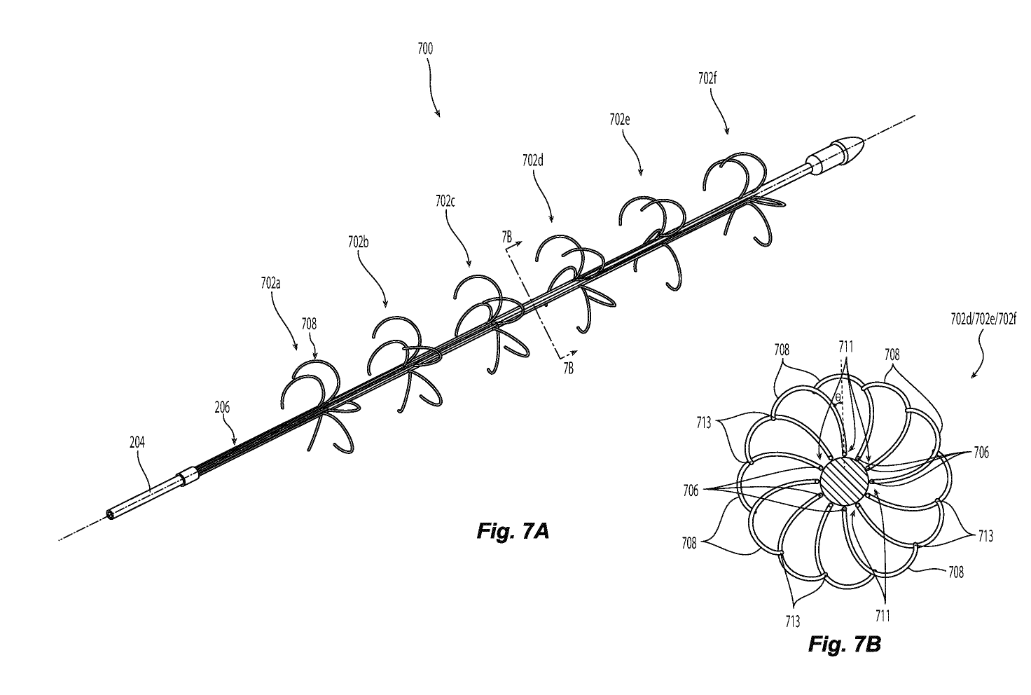

FIG. 7A is a perspective view of another embodiment of a clot treatment device 700 in a deployed state in accordance with the present technology, and FIG. 7B is a cross-sectional end view taken along line 7B-7B in FIG. 7A. Referring to FIGS. 7A and 7B together, the clot treatment device 700 can have groups of clot engagement members 702a-f spaced along the support member 204. The groups 702a-f can include a plurality of arcuate clot engagement members 702 generally similar to the clot engagement members 202 and 402 described with reference to FIGS. 2A-4B, except the second portions 708 of the clot engagement members 702 of FIG. 7A extend at an angle from the support member 204 such that the distal ends 713 of the second portions 708 are not circumferentially aligned with the corresponding proximal ends 711 of the second portions 708. For example, as shown in FIG. 7B, the second portions 708 can extend at an angle θ from the first portions 706. In some embodiments, the angle θ can be between about 10 and about 80 degrees. In a particular embodiment, the angle θ can be between about 40 and about 60 degrees. Additionally, as shown in FIGS. 4B and 7B, the clot engagement members may form a substantially circular axial array about the axis of the support member. A circular array may engage clot more uniformly and securely than a non-circular array and thus may facilitate retrieval and removal of clot from the vessel.

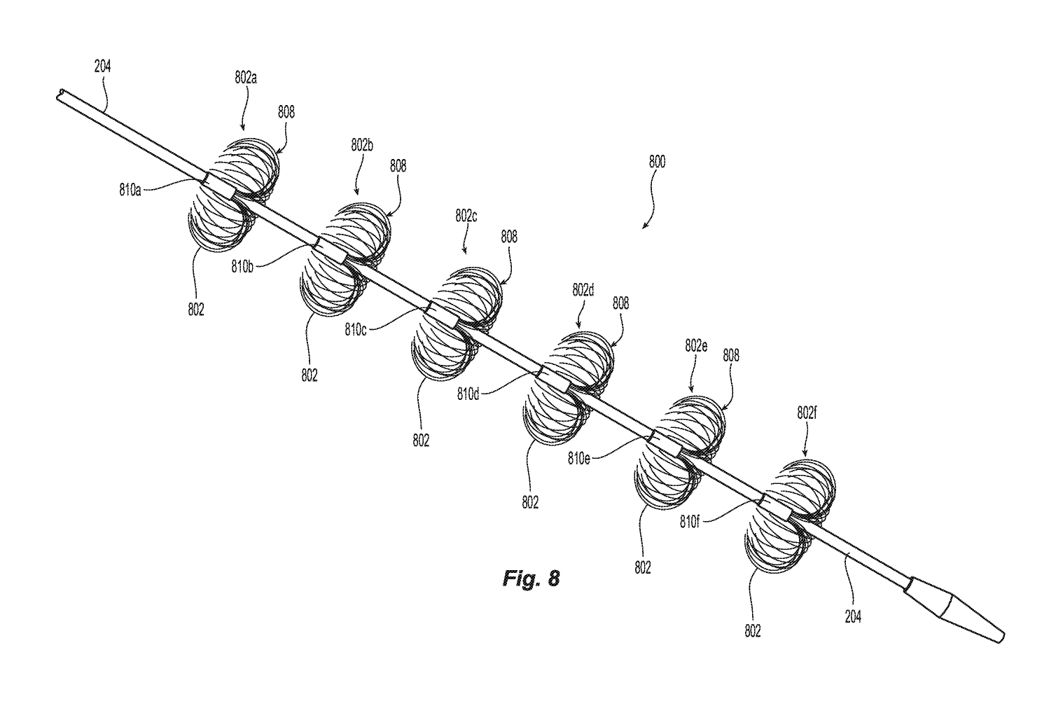

FIG. 8 is a perspective view of another embodiment of a clot treatment device 800 in a deployed state in accordance with the present technology. As shown in FIG. 8, the clot treatment device 800 can have groups of clot engagement members 802a-f spaced along the support member 204. The groups 802a-f can include a plurality of arcuate clot engagement members 802 generally similar to the clot engagement members 202 and 402 described with reference to FIGS. 2A-4B, except the clot engagement members 802 of FIG. 8 do not include a first or cantilevered portion. As such, the clot engagement members 802 include only a curved second portion 808 which is coupled to the support member 204 at one end (e.g., via hubs 810a-f). In a particular embodiment, the clot engagement members 802 can have a first portion; however, in such embodiments, the first portions of the clot engagement members 802 are relatively short (e.g., less than about 10 mm). In some embodiments, the groups 802a-f can be evenly spaced along the support member 204, and in other embodiments the groups 802a-f can have any spacing or state along the support member 204. Additionally, the arcuate clot engagement members 802 at one group 802 can have a different size than the arcuate clot engagement members 802 at a different group 802. The groups 802a-f can be deployed or expanded simultaneously (e.g., via a push-wire or other deployment methods) or consecutively (e.g., by retracting a sheath).

FIG. 9A is a perspective view of another embodiment of a clot treatment device 1200 in a deployed state configured in accordance with the present technology. In some embodiments, the device 1200 can include a plurality of clot engagement members 1202 arranged in closely-packed circular array. The clot engagement members 1202 can be generally similar to the clot engagement members 202 and 402 described with reference to FIGS. 2A-4B. A proximal portion of the clot engagement members 1202 can be bound together and surrounded by a tubular binding member 1210. The clot engagement members 1202 can fill substantially all of a lumen of the binding member 1210, as shown in the cross-sectional view of FIG. 9B (other than the small gaps between the clot engagement members (that are too small for another clot engagement member)). In another embodiment (not shown), a lumen or tube may provide for passage of a guidewire or catheter through the bundle of clot engagement members. Referring to FIG. 9A, the clot engagement members 1202 can have first portions 1206 with differing lengths so that the second portions 1206 are spread out over a deployed engagement member length L. In some embodiments, the deployed engagement member length L may be between about 0.5 cm and about 8 cm, and in some embodiments, between about 1 cm and about 5 cm. As shown in FIG. 9C, the binding member 1210 can be a coil, spiral, tube, sleeve, braid and/or other generally suitable tubular configurations. The binding member 1210 may be slotted, cut or otherwise fenestrated to enhance flexibility. The binding member 1210 may be made of various metals, polymers and combinations thereof and may comprise materials visible under x-ray or fluoroscopy so as to function as a radiopaque marker to facilitate deployment, placement and retraction by the user.

### II. Delivery Systems and Methods

FIG. 10 is a side partial cross-sectional view of one embodiment of a delivery system 910 for delivering the clot treatment device 200 to a treatment site, such as a pulmonary embolism. The delivery system 910 can include a proximal portion 911, an elongated delivery catheter 920 extending from a distal region of the proximal portion 911, a delivery sheath 930 slidably received within a lumen of the delivery catheter 920, a tubular push member 940 slidably received within a lumen of the delivery sheath 930, and a guidewire 912 slidably received within a lumen of the push member 940. As shown in FIG. 10, the clot treatment device 200 can be positioned within the delivery sheath 930 such that the delivery sheath 930 constrains the clot engagement members 202 in a low-profile delivery state that is generally parallel with the support member 204. In some embodiments, the delivery catheter 920 can have an outside diameter between about 0.8 mm and about 1.8 mm, and in some embodiments, between about 0.1 mm and about 0.16 mm. A proximal portion of the support member 204 can be coupled to a distal region of the push member 204 such that axial movement of the push member 204 causes axial movement of the support member 204 (and thus the clot treatment device 200).

The proximal portion 911 of the device can include a first hub 922 and a second hub 932 configured to be positioned external to the patient. The first and/or second hubs 922, 932 can include a hemostatic adaptor, a Tuohy Borst adaptor, and/or other suitable valves and/or sealing devices. A distal region 920a of the first hub 922 can be coupled to the delivery catheter 920, and a proximal region of the first hub 922 can include an opening 924 configured to slidably receive the delivery sheath 930 therethrough. In some embodiments, the first hub 922 can further include an aspiration line 926 coupled to a negative pressure-generating device 928 (shown schematically), such as a syringe or a vacuum pump. A distal region 932a of the second hub 932 can be fixed to a proximal region of the delivery sheath 930, and a proximal region of the second hub 932 can include an opening 934 configured to receive the push member 940 therethrough. Additionally, in some embodiments, the second hub 932 can include a port 936 configured to receive one or more fluids before, during and/or after the procedure (e.g., contrast, saline, etc.).

FIGS. 11A-11K illustrate one example for treating an embolism (e.g., a pulmonary embolism) with the clot treatment device 200 (and delivery system 910). FIG. 11A is a side view of a delivery system 910 positioned adjacent to an embolism or clot material PE within a pulmonary blood vessel V. Access to the pulmonary vessels can be achieved through the patient's vasculature, for example, via the femoral vein. The delivery system 910 can be guided through the right atrium, through the tricuspid valve, into the right ventricle, through the pulmonary valve and into the main pulmonary artery. Depending on the location of the embolism, the delivery system 910 can be guided to one or more of the branches of the right pulmonary artery and/or the left pulmonary artery. It will be understood, however, that other access locations into the venous circulatory system of a patient are possible and consistent with the present technology. For example, the user can gain access through the jugular vein, the subclavian vein, the brachial vein or any other vein that connects or eventually leads to the superior vena cava. Use of other vessels that are closer to the right atrium of the patient's heart can also be advantageous as it reduces the length of the instruments needed to reach the pulmonary embolism.

As shown in FIG. 11A, the delivery sheath 930 containing the collapsed clot treatment device 200 (not shown) can be advanced together with the delivery catheter 920 over the guidewire 912 to the treatment site. For example, the guidewire 912 can be inserted through the target pulmonary embolism PE. Referring to FIG. 11B, a distal portion of the delivery catheter 920 and/or delivery sheath 930 can then be advanced through the pulmonary embolism PE such that the distal ends 201 of at least one group of the clot engagement members 202 are aligned with or positioned distal to a distal edge of the pulmonary embolism PE. In other embodiments (not shown), a distal portion of the delivery catheter 920 and/or delivery sheath 930 can be positioned such that the distal ends 201 of at least one group of the clot engagement members 202 are positioned proximal to a distal edge of the pulmonary embolism PE.

Once the device is positioned, the guidewire 912 can then be removed proximally through a lumen of the delivery sheath 930 and/or delivery catheter 920, and the delivery sheath 930 can be pulled proximally to a position proximal of the pulmonary embolism PE (as shown in FIG. 11B). As shown in FIGS. 11C-11G, the delivery sheath 930 can be retracted proximally to expose the distal portions of the second portions 208 of the clot engagement members such that the exposed portions radially expand and bend backwards in a proximal direction. As the second portions 208 expand, they extend into the pulmonary embolism PE around the device along an arcuate path P. The arcuate path P can extend radially outward and proximally with respect to the support member (not shown) and, as shown in FIG. 11F, can eventually curve radially inwardly. The second portions 208 can thus form hook-like capture elements that penetrate into and hold clot material to the device 200 for subsequent removal. Moreover, should the second portions 208 extend radially outwardly enough to touch the vessel wall, the end sections 214 of the second portions 208 form an atraumatic surface that can abut or apply pressure to the vessel wall without damaging the vessel wall. In some embodiments, the device presents a plurality of arcuate members that may be substantially parallel with the axis of the device at the point of contact with the vessel wall when in the deployed state.

Still referring to FIG. 11F, when the delivery sheath 930 is withdrawn proximally beyond the second portions 208 of the most distal group of clot engagement members 202f, the first portions 206 of the clot engagement members 202f are exposed. In some embodiments, the delivery sheath 930 can be withdrawn so as to expose only a portion of the clot engagement members. Additionally, in those embodiments having two or more groups of clot engagement members, the delivery sheath 930 can be withdrawn to expose all or some of the groups of clot engagement members. As shown in FIG. 11G, the delivery sheath 930 can continue to be withdrawn proximally to expose additional second portions 208 and/or groups of clot engagement members 202a-f. Clot engagement members 202a-f may just contact or be slightly deflected by the vessel wall. If the device is sized such that the diameter of the clot engagement members are larger than the vessel diameter (e.g., “over-sized”), the clot engagement members may be compressed by the vessel wall. Thus, while fully deployed, the device may be in state of a small amount of radial compression. In some embodiments, the device may be diametrically over-sized by between about 5% and 50% and in other embodiments between about 10% and 25%.

As shown in FIGS. 11H-11K, once at least a portion of the clot engagement members and/or second portions 208 have penetrated and engaged the targeted clot material PE, the clot treatment device 200 can be withdrawn proximally, thereby pulling at least a portion of the clot material PE in a proximal direction with the device 200. For example, the push member 940, second hub 932, and delivery sheath 930 (FIG. 10) can be retracted proximally at the same time and rate. As such, the delivery catheter 920 can be held in place while the delivery sheath 930, clot material PE, and clot engagement device 200 are pulled proximally into the delivery catheter 920. The curved shape of the second portions 208 increases the surface area of the clot engagement members 202 in contact with the clot material PE, thus increasing the proximal forces exerted on the clot material. Withdrawal of the device 200 not only removes the clot but also can increase blood flow through the vessel.

As shown in FIG. 11K, in some embodiments the delivery catheter 920 can include an aspiration lumen (not shown) configured to apply a negative pressure (indicated by arrows A) to facilitate removal of the clot material PE. For example, the delivery catheter 920, delivery sheath 930 and/or clot treatment device 200 of the present technology can be configured to be operably coupled to the retraction and aspiration apparatus disclosed in Attorney Docket No. 111552.8004.US00, titled “Retraction and Aspiration Apparatus and Associated Systems and Methods,” filed concurrently herewith, which is incorporated herein by reference in its entirety. When coupled to the retraction and aspiration apparatus, a negative pressure is applied at or near the distal portion of the delivery catheter 920 (via the aspiration lumen) only while the clot treatment device 200 and/or delivery sheath 930 is being retracted. Therefore, when retraction pauses or stops altogether, aspiration also pauses or stops altogether. Accordingly, aspiration is non-continuous and dependent upon retraction of the delivery sheath 930 and/or clot treatment device 200. Such non-continuous, synchronized aspiration and retraction can be advantageous because it reduces the amount of fluid withdrawn from the patient's body during treatment (and thus less fluid need be replaced, if necessary). In addition, it may be advantageous to consolidate the steps and motions required to both mechanically transport thrombus into the guide catheter (e.g. aspiration tube) and remove fluid from the tube into one motion, by one person.

Although the invention has been described in terms of particular embodiments and applications, one of ordinary skill in the art, in light of this teaching, can generate additional embodiments and modifications without departing from the spirit of or exceeding the scope of the exampled invention. Accordingly, it is to be understood that the drawings and descriptions herein are proffered by way of example to facilitate comprehension of the invention and should not be construed to limit the scope thereof.

1. A clot treatment device for treating a pulmonary embolism within a blood vessel, the clot treatment device moveable between a low-profile undeployed state and a deployed state, the clot treatment device comprising:
a support member configured to extend through a delivery catheter, wherein the support member has a proximal portion and a distal portion;
a plurality of clot engagement members positioned about the circumference of the distal portion of the support member, wherein individual clot engagement members have a curved portion;
wherein, in the deployed state, individual curved portions project radially outwardly relative to the support member in a curve that has a proximally extending section which defines a proximally facing concave portion, and wherein the curved portion further includes an end section that curves radially inwardly from the proximally extending section; and
wherein the clot engagement members are configured to penetrate clot material along an arcuate path and hold clot material to the clot treatment device.

2. The clot treatment device of claim 1 wherein individual clot engagement members further include a cantilevered portion having a proximal region attached to the support member and a distal region, and wherein the cantilevered portion extends distally in a longitudinal direction from the proximal region to the distal region.

3. The clot treatment device of claim 2 wherein individual curved portions extend from the distal region of one of the cantilevered portions.

4. The clot treatment device of claim 1 wherein individual curved portions have a first region with a first radius of curvature and a second region with a second radius of curvature different than the first radius of curvature.

5. The clot treatment device of claim 1 wherein individual curved portions have a first curved section that extends distally and a second curved section that extends proximally.

6. The clot treatment device of claim 1 wherein the plurality of clot engagement members includes a plurality of first clot engagement members and a plurality of second clot engagement members.

7. The clot treatment device of claim 1 wherein the plurality of clot engagement members includes a plurality of first clot engagement members and a plurality of second clot engagement members, each having a curved portion.

8. The clot treatment device of claim 1 wherein the plurality of clot engagement members includes a plurality of first clot engagement members and a plurality of second clot engagement members, each having a curved portion and a cantilevered portion, and wherein—
the cantilevered portions of the first clot engagement members have a first length; and
the cantilevered portions of the second clot engagement members have a second length different than the first length.

9. The clot treatment device of claim 1 wherein the plurality of clot engagement members includes a plurality of first clot engagement members and a plurality of second clot engagement members, each having a curved portion and a cantilevered portion, and wherein—
the cantilevered portions of the first clot engagement members have a first length;
the cantilevered portions of the second clot engagement members have a second length different than the first length;
the curved portions of the second clot engagement members are circumferentially offset from the curved portions of the first clot engagement members.

10. The clot treatment device of claim 1, wherein the plurality of clot engagement members are substantially linear and parallel with the support member when in the low-profile undeployed state.

11. A clot treatment device for treating a pulmonary embolism, the clot treatment device moveable between a low-profile undeployed state and a deployed state, the clot treatment device comprising:
a support member configured to extend through a delivery catheter, wherein the support member has a proximal portion and a distal portion;
a plurality of clot engagement members positioned about the circumference of the distal portion of the support member, wherein individual clot engagement members have a first portion and a second portion extending from the first portion, and wherein—
individual first portions have a proximal region attached to the support member and a distal region, and wherein the first portions extend distally in a longitudinal direction from the proximal region to the distal region; and
in the deployed state, individual second portions extend from the distal region of one of the first portions, and wherein individual second portions project radially outwardly relative to the support member in a curve that has a proximally extending section which defines a proximally facing concave portion; and
the clot engagement members are configured to penetrate clot material along an arcuate path and fasten clot material to the clot treatment device.

12. The clot treatment device of claim 11 wherein each second portion further includes an end section curving radially inward from the proximally extending section.

13. The clot treatment device of claim 11 wherein the first portions of the clot engagement members are cantilevered portions.

14. The clot treatment device of claim 11 wherein individual second portions have a first region with a first radius of curvature and a second region with a second radius of curvature different than the first radius of curvature.

15. The clot treatment device of claim 11 wherein individual second portions have a first curved section that extends distally and a second curved section that extends proximally.

16. The clot treatment device of claim 11 wherein the plurality of clot engagement members includes a plurality of first clot engagement members and a plurality of second clot engagement members.

17. The clot treatment device of claim 11 wherein the plurality of clot engagement members has a total number between 12 and 60.

18. The clot treatment device of claim 11 wherein the plurality of clot engagement members includes a plurality of first clot engagement members and a plurality of second clot engagement members, each having a first portion and a second portion, and wherein—
the first portions of the first clot engagement members have a first length; and
the first portions of the second clot engagement members have a second length different than the first length.

19. The clot treatment device of claim 11 wherein the plurality of clot engagement members includes a plurality of first clot engagement members and a plurality of second clot engagement members, each having a first portion and a second portion, and wherein—
the first portions of the first clot engagement members have a first length;
the first portions of the second clot engagement members have a second length different than the first length;
the second portions of the second clot engagement members are circumferentially offset from the second portions of the first clot engagement members.

20-27. (canceled)
